(19) United States
(12) Patent Application Publication
(10) Pub. No.: US 2007/0129554 A1
(43) Pub. Date: Jun. 7, 2007

(54) Alpha functionalization of cyclic, ketalized ketones and products therefrom

(76) Inventors: Peter J. Harrington, Louisville, CO (US); Hiralal N. Khatri, Louisville, CO (US); Sudha Khatri, Louisville, CO (US)

Correspondence Address: Roche Colorado Corporation, 2075 North 55th Street, Boulder, CO, 80301, US

(21) Appl. No.: 11/583,971

(22) Filed: Oct. 19, 2006

Related U.S. Application Data
(60) Provisional application No. 60/729,955, filed on Oct. 24, 2005.

(51) Int. Cl. C07D 313/20; C07D 313/06

(52) U.S. Cl. 549/331

(57) ABSTRACT

Methodologies for the alpha-monohalogenation of acid sensitive ketones, especially cyclic, acid-sensitive, ketalized ketones. As one approach, the ketone is reacted with a halogen donor compound, e.g., N-chlorosuccinimide, in anhydrous, highly polar organic reagents such as dimethylformamide (DMF). As another monohalogenation approach, it has been observed that organic salts generated from amines and carboxylic acids catalyze the monohalogenation of ketalized ketone in reagents comprising alcohol solvent (methanol, ethanol, isopropanol, etc.). The monohalogenation is fast even at −5° C. The salt can be rapidly formed in situ from ingredients including amines and/or carboxylic acids without undue degradation of the acid sensitive ketal. Aryl ketones are monooxygenated using iodosylbenzene. This methodology is applied to monohalogenation of an acid sensitive monoketal ketone. The ability to prepare monohalogenated, acid sensitive ketones facilitates syntheses using halogenated, acid sensitive ketones. As just one example, facile synthesis of halogenated, acid sensitive ketones provides a new approach to synthesize the S-ketal-acid S-MBA (S-methylbenzylamine) salt useful as an intermediate in the manufacture of a glucokinase activator. As an overview of this scheme, a monohalogenated, cyclic, ketalized ketone is prepared using monohalogenation methodologies of the present invention. The halogenated compound is then subjected to a Favorskii rearrangement under conditions to provide the racemic acid counterpart of the desired chiral salt. The desired chiral salt is readily recovered in enantiomerically pure form from the racemic mixture.

### PRIORITY CLAIM

The present non-provisional patent Application claims priority under 35 USC §119(e) from United States Provisional Patent Application having serial number 60/729,955, filed on Oct. 24, 2005, by Harrington et al. and titled ALPHA FUNCTIONALIZATION OF CYCLIC, KETALIZED KETONES AND PRODUCTS THEREFROM, wherein the entirety of said provisional patent application is incorporated herein by reference for all purposes.

### BACKGROUND OF THE INVENTION

The glucokinase activator 70 shown in FIG. 9 is under evaluation in Phase I clinical studies as a potentially new therapy for the treatment of Type 2 diabetes. This compound has also been described in PCT Patent Publication No. WO 03/095438. An important intermediate involved in the synthesis of this activator is a chiral salt, specifically, an S-ketal-acid S-MBA (S-methylbenzylamine) salt having following structure: 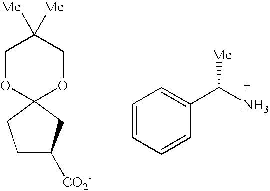
Previous routes to this intermediate have proceeded through the ketalization of 3-oxo-1-cyclopentanecarboxylic acid according to the scheme shown in FIG. 1a (prior art). It would be desirable to provide a route to this chiral salt that offers higher throughput. The conventional scheme also suffers from waste issues. Specifically, the keto acid precursor of the salt is highly soluble in water. In order to accomplish workup and isolation, relatively large amounts of salt, e.g., sodium sulfate, are added. This makes the aqueous solution sufficiently ionic so that the oxocyclopentane carboxylic acid can be extracted into an organic solvent. As much as 5 to 6 parts by weight of salt per part by weight of compound may be required to accomplish this. In the end, the salt must be handled as waste. It would be highly desirable to provide a synthesis that reduces or even avoids such waste issues.

The α-halogenation of a ketone is known. Since the reaction is believed to proceed via the enol, it is often base or acid-catalyzed. However, base catalysis usually results in polychlorination. Acid catalysis, therefore, is preferable when a monohalogenated ketone is desired.

However, when a ketone includes a ketal or acetal moiety, the presence of the acid catalyst causes degradation of the reactant and/or halogenated product, e.g., loss, of the ketal moiety. Thus, the monohalogenation of a cyclic, ketalized ketone such as the 1,4-cyclohexanedione mono(2,2-dimethyltrimethylene ketal) shown in FIG. 3 has been quite difficult.

The monochlorination of tetrahydropyran-4-one with NCS in dichloromethane and acid-base catalyzed monochlorination of 1,4-cyclohexanedione monoethylene acetal with NCS in acetonitrile have been recently described. See Marigo, M.; Bachmann, S.; Halland, N.; Braunton, A.; Jorgensen, K. A. (2004) Angew. Chem. Int. Ed. Engl., 43:5507.

The combination of NCS-DMF has been used for chlorination of aldoximes, Liu, K- C.; Shelton, B. R.; Howe, R. K. (1980) J Org. Chem. 45:3916, and of aromatics, Wilkerson, W. W. U.S. Pat. No. 4,652,582 (Mar. 24, 1987).

It has recently been reported that aldehyde and ketone chlorinations can be catalyzed by organic salts generated from amines and carboxylic acids. Marigo, M.; Bachmann, S.; Halland, N.; Braunton, A.; Jorgensen, K. A. (2004) Angew. Chem. Int. Ed. Engl.: 5507. Halland, N.; Braunton, A.; Bachmann, S.; Marigo, M.; Jorgensen, K. A. (2004) J Amer. Chem. Soc. 126:4790. Brochu, M. P.; Brown, S. P.; MacMillan, D. W. C. (2004) J Amer. Chem. Soc. 126:4790. It also is known that aryl ketones can be monooxygenated using iodosylbenzene. Handbook of Reagents for Organic Synthesis: Oxidizing and Reducing Agents, S. D. Burke and R. L. Danheiser, eds., John Wiley & Sons, New York, 1999, pp. 122-125. The iodosylbenzene is generated from diacetoxyiodobenzene with potassium hydroxide in methanol at 25° C. Under the same conditions (PhI(OAc)₂, KOH, CH₃OH), there are two examples where a cyclohexanone undergoes monofunctionalization and rearrangement to produce cyclopentanecarboxylic acid in a single operation. Daum, S. J. (1984) Tetrahedron Lett. 25:4725; Iglesias-Arteaga, M. A.; Velazquez-Huerta, G. A. (2005) Tetrahedron Lett. 46:6897.

### SUMMARY OF THE INVENTION

The present invention provides methodologies for the alpha-monohalogenation of acid sensitive ketones, especially cyclic, acid-sensitive, ketalized ketones. As one approach, the ketone is reacted with a halogen donor compound, e.g., N-chlorosuccinimide, in anhydrous, highly polar organic reagents such as dimethylformamide (DMF). The reaction is clean and occurs with high yield, showing high selectivity for the desired monohalogenated ketone. By-products associated with base catalysis and ketal degradation associated with acid catalysis are substantially avoided.

As another monohalogenation approach, it has been observed that organic salts generated from amines and carboxylic acids catalyze the monohalogenation of ketalized ketone in reagents comprising alcohol solvent (methanol, ethanol, isopropanol, etc.). The monohalogenation is fast even at −5° C. The salt can be rapidly formed in situ from ingredients including amines and/or carboxylic acids without undue degradation of the acid sensitive ketal. The suspension of the resultant halogenated ketone in alcohol can be transferred directly to further processing, e.g., a Favorskii rearrangement.

As noted above, it is known that aryl ketones can be monooxygenated using iodosylbenzene. This methodology may be very efficiently applied to monohalogenation of an acid sensitive monoketal ketone and is especially useful to provide iodine (e.g., in a higher oxidation state) as the leaving group.

The ability to prepare monohalogenated, acid sensitive ketones has also facilitated syntheses using halogenated, acid sensitive ketones. As just one example, facile synthesis of halogenated, acid sensitive ketones provides a new approach to synthesize the S-ketal-acid S-MBA (S-methylbenzylamine) salt useful as an intermediate in the manufacture of the glucokinase activator 70 shown in FIG. 9. As an overview of this scheme, which is shown in FIG. 1b, a monohalogenated, cyclic, ketalized ketone is prepared using monohalogenation methodologies of the present invention. The halogenated compound is then subjected to a Favorskii rearrangement under conditions to provide the racemic acid counterpart of the desired chiral salt. The desired chiral salt is readily recovered in enantiomerically pure form from the racemic mixture.

For instance, as shown in FIG. 1b, the 2-chlorocyclohexanone may be prepared via mono-alpha-chlorination of a commercially available 1,4-cyclohexanedione mono(2,2-dimethyltrimethylene ketal). The halogenated 1,4-cyclohexanedione mono(2,2-dimethyltrimethylene ketal) is subjected to a Favorskii rearrangement to give 8,8-dimethyl-6,10-dioxaspiro[4,5]decane-2-carboxylic acid. This product is then converted to the S-MBA salt.

In one aspect, the present invention relates to a compound, comprising a cyclic moiety comprising a backbone of at least 4 atoms and having first and second alpha positions adjacent a keto group; at least one hydrogen substituent positioned at the first alpha position; a leaving group substituent positioned at the second alpha position; and a ketal substituent positioned at a third position that is at a beta position or further from the keto group.

In another aspect, the present invention relates to a method of alpha-halogenating a ketone compound is provided. The ketone compound comprises a cyclic moiety comprising a backbone of at least 4 atoms and having first and second alpha positions adjacent a keto group; at least one hydrogen substituents positioned at the first alpha position; a leaving group substituent positioned at the second alpha position; and a ketal or acetal substituent positioned at a third position that is at a beta position or further from the keto group. A halogen donor compound also is provided. Ingredients including the ketone compound and the donor compound are reacted in a substantially anhydrous solvent that is sufficiently polar so that alpha-functionalization of the keto compound occurs.

In another aspect, the present invention relates to a method of halogenating a ketalized ketone. The ketone is halogenated in an anhydrous, organic reagent in the presence of a salt catalyst, wherein the reagent comprises an alcohol.

In another aspect, the present invention relates to a method of making a ketal acid comprising reacting a ketalized ketone with an iodine donor compound in an alkaline reaction medium.

In another aspect, the present invention relates to a method of making a compound. A ketalized, cyclic ketone is halogenated at an alpha position relative to a keto group. The halogenated, ketalized cyclic ketone is subjected to a ring contraction reaction.

### BRIEF DESCRIPTION OF THE DRAWINGS

FIG. 1a shows a prior art reaction scheme for preparing an (S)-MBA salt.

FIG. 1b shows a reaction scheme of the present invention for preparing an (S) MBA salt.

FIG. 2 shows a formulae of cyclic, ketalized ketones useful in practicing aspects of the present invention.

FIG. 3 shows a preferred embodiment of a cyclic, ketalized ketone.

FIG. 4 shows an illustrative reaction scheme for preparing the compound of FIG. 3.

FIG. 5 shows an illustrative reaction scheme for functionalizing the cyclic, ketalized ketone of FIG. 3 at an alpha position relative to the ketone moiety.

FIG. 6 schematically illustrates how a Favorskii rearrangement reaction is carried out.

FIG. 7 schematically illustrates how a Favorskii rearrangement reaction is carried out with respect to a cyclic, ketalized, alpha-functionalized ketone.

FIG. 8 schematically shows one approach for obtaining an enantiomerically pure chiral (S) salt intermediate from a cyclic, ketalized ketone.

FIG. 9 shows the chemical structure of a glucokinase activator of the prior art.

### DETAILED DESCRIPTION

In one aspect, the present invention relates to the alpha functionalization of acid sensitive ketones such as a cyclic, ketalized ketone. A cyclic, ketalized ketone generally includes a cyclic moiety incorporating a keto group, —C(O)—, and comprising a backbone of at least 4 atoms, typically 4 to 8, preferably 5 or 6, most preferably 6 atoms. The keto group may be part of the backbone or may be part of a substituent pendant from the backbone, but preferably is part of the backbone. The backbone atoms may include C, O, N, S, combinations of these and the like. The cyclic backbone may be saturated or unsaturated, but preferably is saturated. A preferred backbone is formed from carbon atoms, e.g., C1-C5 or C1-C6 structures. For reference purposes, the keto group may be deemed to be associated with the C1 carbon.

The ketalized character of the ketone means that the molecule incorporates a ketal moiety, e.g., as a portion of the backbone or as part of a substituent that is pendant from the backbone. The cyclic, ketalized ketone used in the present invention includes at least one ketal moiety positioned at a beta position or further from the keto group. Thus, for a six-membered cyclic structure in which the keto moiety is at the C1 position, the ketal group may be at the C3, C4, or C5 position. A ketal group at the C4 position is preferred. The ketal group desirably is not at either alpha position relative to the keto group, e.g., at either the C2 or C6 position in the case of a six-membered ring, so as not to interfere with alpha functionalization or subsequent Favorskii rearrangement in some embodiments.

A ketal is a functional group, or a molecule containing the functional group, of a carbon atom bonded to both —OZ¹ and —OZ² groups, wherein each of Z¹ and Z² independently may be a wide variety of monovalent moieties or co-members of a ring structure. A ketal is structurally equivalent to an acetal, and sometimes the terms are used interchangeably. In some uses, a difference between an acetal and a ketal derives from the reaction that created the group. Acetals traditionally derive from the reaction of an aldehyde and excess alcohol, whereas ketals traditionally derive from the reaction of a ketone with excess alcohol. For purposes of the present invention, though, the term ketal refers to a molecule having the resultant ketal/acetal structure regardless of the reaction used to form the group.

To facilitate alpha functionalization, the ketalized ketone desirably includes at least one H atom at one of the alpha positions relative to the keto group. Preferably, at least one H atom is also present at the other alpha position, especially in those embodiments in which the alpha-functionalized product is used in a subsequent Favorskii rearrangement, described further below. Most preferably, each alpha position bears only H substituents.

In addition to the keto group, ketal group, and alpha hydrogen(s), the cyclic backbone of the cyclic, ketalized ketone may include one or more other substituents. Generally, these other substituents may be selected so as to be relatively nonreactive under the conditions used for alpha-functionalization to minimize the formation of undesirable by products. Additionally, when the resultant alpha-functionalized product is subsequently subjected to a Favorskii rearrangement, it is desirable that the other substituents also be selected so as to be relatively nonreactive under the conditions used for the rearrangement. With these concerns in mind, examples of other substituents that may be present include hydrogen; linear, branched, or cyclic alkyl; alkoxy, aryl, combinations of these and the like. Hydrogen and lower alkyl of 1 to 4 carbon atoms are preferred. Examples of other substituents that desirably are avoided in some modes of practice, especially when a Favorskii rearrangement is contemplated, include ketones, nitro groups, aldhehyde moieties, or other ketone reactive groups such as groups that may be deprotonated and/or condense with a ketone, and the like. A review of the scope and limitations of a Favorskii Rearrangement is provided in Organic Reactions, 11:261-316 (1960).

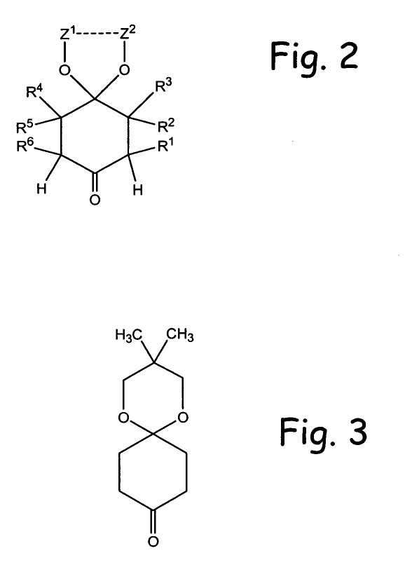

Preferred embodiments of the cyclic ketalized ketone are represented by the formula shown in FIG. 2, wherein each of Z¹ and Z² independently represents a monovalent group, or as represented by a dashed line, are co-members of a ring structure providing a divalent moiety -Z¹-Z²-. In representative embodiments, Z¹ and Z² alone or as co-members of a ring structure are linear, branched, or cyclic alkyl(ene); preferably alkyl(ene) of 1 to 15, preferably 2 to 5 carbon atoms. The divalent, branched alkylene backbone associated with neopentyl glycol is a preferred structure when Z¹ and Z² are co-members of a ring structure.

Each of the R1 through R6 substituents independently represents a monovalent group such as those selected from hydrogen; linear, branched, or cyclic alkyl; alkoxy, aryl, combinations of these, and the like. Any two or more of the R¹ through R⁶ substituents also may be co-members of a ring structure. Preferably, R¹ through R⁶ are hydrogen. When the alpha-functionalized product is to be subjected to a Favorskii rearrangement, it is desirable that none of the R¹ through R⁶ substituents be selected from ketones, nitro groups, moieties that may be deprotonated and/or condense with a ketone, and the like, as such groups tend to be unduly reactive under the Favorskii rearrangement conditions.

Note that the compounds of FIG. 2 are based upon a 6-membered ring backbone and include at least one H substituent at an alpha position relative to the keto group, more preferably at least one H substituent at each alpha position relative to the keto group.

A particularly preferred example of a compound according to FIG. 2 is shown in FIG. 3. This compound is preferred for a number of reasons. First, it is a symmetric molecule, and thus alpha-functionalization occurs with higher yield and with a lesser number of functionalized by-products as might otherwise occur if the ketal group were to be asymmetrically positioned relative to the keto group, e.g., at the C3 or C5 position for instance. Also, this compound is not only commercially available, but literature methods for its synthesis from widely available materials are also known.

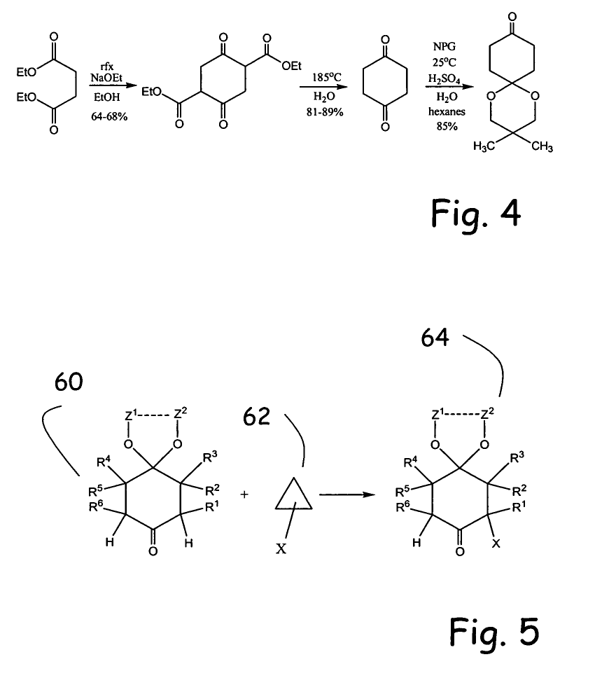

One representative reaction scheme for forming the compound of FIG. 3 from commodity chemicals is shown in FIG. 4. In a first step, diethyl succinate is essentially dimerized by heating at reflux in anhydrous ethanol in the presence of NaOEt. This compound is then heated in water to produce the 1,4-cyclohexanedione. The dione is then reacted with neopentyl glycol (NPG) in acidic, aqueous solution in the presence of a hexane phase to form the monoketal. The monoketal is soluble in the hexane and tends to go to that phase to avoid forming the diketal. The reaction steps used in the scheme of FIG. 4 are known and described in the literature. For instance, the monoketalization of 1,4-cyclohexanedione is described in Babler, J. H.; Spina, K. P. (1984) Synth. Commun. 14:39; and Reguri, B. R.; Kadaboina, R.; Gade, S. R.; Ireni, B. I. U.S. Pat. Pub. No. 2004/0230063 (Nov. 18, 2004). The preparation of 1,4-cyclohexanedione from diethyl succinate is described in Nielsen, A. T.; Carpenter, W. R. (1965) Org. Syntheses 45:25.

When the monoketal ketone of FIG. 3 is synthesized using illustrative processes as described and/or referenced herein, a bisketal by-product may tend to be formed. It is desirable to separate the monoketal from the bisketal at as high a purity as is practical. Conventional techniques might allow recovery of the monoketal ketone at a purity of 95% by weight with respect to the bisketal by-product. An illustrative extraction process described herein may be used to obtain highly pure monoketal ketone from a monoketal-bisketal mixture such as that obtained by Babler, J. H.; Spina, K. P. (1984) Synth. Commun. 14:39. This isolation process (see Experimental section, Example 1) facilitated the separation of the monoketal and bisketal without resorting to fractional distillation under high vacuum (<1 mm Hg) and provides monoketal ketone at a purity of over 99%. One aspect of the purification process described herein involves using the right kind of solvents for extraction, preferably at the right ratios.

The present invention provides a very clean alpha functionalization of the cyclic, ketalized ketones. In preferred embodiments, the alpha-functionalization is an alpha-halogenation, more preferably an alpha-mono-halogenation with no addition of a catalyst being required, because a suitable catalytic agent is believed to form in situ. Alpha-halogenation refers to functionalizing the cyclic, ketalized ketone with Cl, Br, and/or I, although Cl is most economical presently. When the resultant alpha-functionalized product is to be subjected to a subsequent Favorskii reaction, the alpha halogen substituent functions as a leaving group. But, halogen is not the only leaving group in the context of the Favorskii rearrangement. Others include alpha-hydroxy (see Craig, J. C.; Dinner, A.; Mulligan (1972) P. J. J. Org. Chem. 37:3539, and the cyclic ketalized ketone may also be alpha-functionalized with any one or more of these other leaving groups as desired.

FIG. 5 shows an illustrative reaction scheme in which a cyclic, ketalized, ketone compound 60 may be reacted with a donor compound 62 serving as a source of the group X to form the mono-alpha-functionalized, cyclic, ketalized ketone product including X as a substituent at an alpha position. In the practice of the present invention, X may be a wide range of functional groups, including Cl, Br, I, OH, combinations of these and the like.

As shown in FIG. 5, the cyclic, ketalized ketone 60 is reacted with a leaving group donor compound 62 in an anhydrous solvent that is sufficiently polar such that alpha functionalization occurs to provide reaction product 64. Generally, if the solvent is insufficiently polar, the reaction may not occur and/or reaction products may be unstable when formed. Examples of an anhydrous solvents found to be sufficiently polar to carry out alpha-chlorination at 25° C. include the highly polar DMF. On the other hand, it was found that dichloromethane and acetonitrile were insufficiently polar when used alone in otherwise similar alpha chlorinations at 25° C.

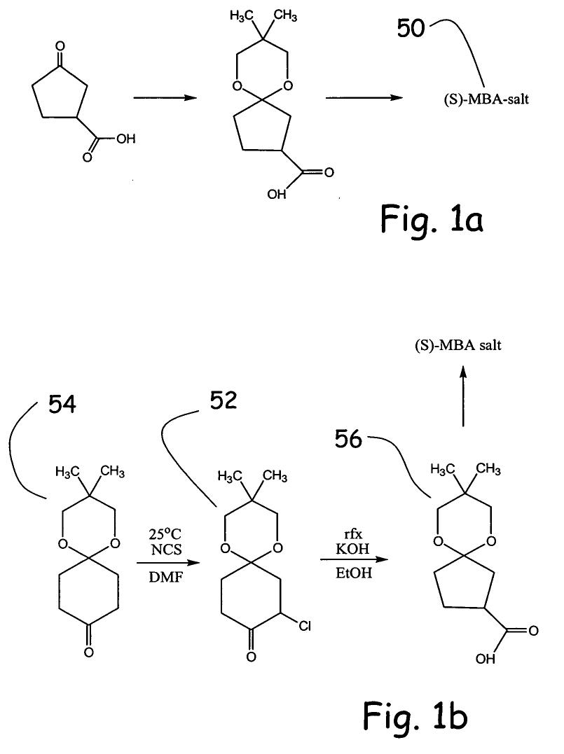

For instance, the monochlorination of a ketalized, ketone according to FIG. 1b with N-chlorosuccinimide (NCS) in dry dimethylformamide, dichloromethane and acetonitrile was evaluated at 25° C. The solvents were substantially fully deuterated (i.e., all H were replaced by deuterium). No reaction of 1 equivalent of the ketone with 1 equivalent NCS was observed in dichloromethane-d2 after 22 h at 25° C. The reaction of the ketone with 1 equivalent NCS in acetonitrile-d3 was faster but still incomplete after 9 days at 25° C. Further, on day ten, significant decomposition of the mixture was observed. It is believed that such decomposition may be catalyzed by hydrogen chloride generated during the course of the reaction and/or during aging. In contrast, the reaction of the ketone with 1 equivalent NCS in dimethylformamide-d7, which is the most polar of the three solvents, was clean and complete in 24-48 h at 25° C. and the product solution remained unchanged after 10 days at 25° C.

Accordingly, the reaction medium used to carry out the reaction scheme of FIG. 5 preferably incorporates at least dry DMF. However, other polar organic solvents such as dichloromethane or acetonitrile might be suitable when used alone or in combination with other reagents in reactions carried out at higher temperatures or otherwise different reaction conditions and/or with different reactants. Additionally, mixtures of DMF with other polar organic solvents such as dichloromethane or acetonitrile would be within the scope of the present invention.

The alpha-functionalization reaction of FIG. 5 desirably is substantially noncatalyzed. Except for reactants themselves, which may be slightly inherently acidic or basic in some embodiments, the alpha functionalization preferably occurs in the substantial absence of added base and acid catalysts or other acidic or basic materials. This helps to avoid generating by-products otherwise associated with basic catalysts and/or ketal degradation otherwise associated with acid catalysts. While it is possible that some moderately acidic or basic species may be generated in the course of the alpha functionalization of the present invention, such species (if any) are not present in amounts that cause undue degradation of the ketal or that unduly impair yield.

The donor compound 62 serves as at least one source of the group(s) to be added to the alpha position of the cyclic, ketalized ketone 60. A wide variety of such compounds are known, and any of these can be used. For alpha-halogenation, preferred donor compounds 62 are those in which the halogens are attached to a nitrogen. These are preferred donor compounds in that the by-product tends to be a neutral compound rather than an acid. For instance, such a donor compound might include the moiety —C(O)—N(X)—, wherein X is a halogen atom. After the functionalization reaction, the moiety might be converted to the more neutral moiety —C(O)—N(H)—. Such donor compounds also desirably are water soluble, and thus are easily separated from the relatively water insoluble, alpha functionalized, cyclic ketalized ketone. In the case of alpha-chlorination, suitable donor compounds 62 in which the chlorine is attached to nitrogen include N-chlorosuccinimide (NCS), dichlorodimethylhydantoin, trichloroisocyanurate, combinations of these, and the like.

The relative amounts of the cyclic, ketalized ketone 60 and donor compound 62 may vary over a wide range. If too little donor compound 62 is used, then incomplete conversion, product mixtures, or the like might result. On the other hand, if too much donor compound 62 is used, then polyfunctionalization might be observed. Often, it is convenient if the reactants 60 and 62 are present in the stoichiometric amount or if there is a very slight stoichiometric excess of functional group. Thus, using 1.25:1, preferably 1.1:1, more preferably 1.05:1 equivalents of the functional group provided on the donor compound 62 to the ketone 60 would be suitable.

The alpha functionalization reaction of FIG. 5 may be carried out at a wide range of temperatures. However, if the temperature is too cool, the reaction may proceed too Generally carrying out the reaction at a temperature in the range of from about −10° C. to about 35° C., preferably about 0 to 25° C. would be preferred.

At least until the reaction is complete, water is desirably excluded as much as is practical from the reaction. Preferred reaction media include less than 1%, preferably less than 0.2, more preferably less than 0.15 weight percent water based upon the total weight of reaction media.

Some of the reactants and/or product may be photosensitive. Thus, it is desirable that the reaction occurs in the substantial absence of ultraviolet light, e.g., in the dark. The optional work up and isolation of the resultant functionalized product also may occur in the absence of ultraviolet light, e.g., the dark, as well.

The reaction of FIG. 5 optionally may occur under ambient atmosphere or in a protected environment, e.g., in an inert atmosphere of one or more gases including nitrogen, argon, helium, carbon dioxide, combinations of these, and/or the like.

Other procedures to carry out alpha-halogenation of an acid sensitive monoketal ketone such as the compound shown in FIG. 3 may also be used in the practice of the present invention. For instance, the halogenation may be carried out in the presence of a salt, which may be formed in situ from one or more suitable precursors and which is believed to catalyze the desired reaction. Preferred salts are formed in situ by incorporating one or more precursors including amine fuctionality and carboxylic acid functionality. When combined in aqueous solution, compound(s) including such functionalities will tend to rapidly form a salt without any undue degradation of the acid sensitive ketal ketone.

As one option, the salt is provided in situ by combining ingredients comprising at least one amine and at least one carboxylic acid. The amine moiety(ies) of the amine may be primary, secondary, or tertiary. Use of a chiral amine may be desired to help form a chiral halogenated product. Examples of suitable amines include simple dialkylamines or cyclic amines of 5- or higher-membered rings amines such as pyrrolidine and imidazolidine, morpholine, piperidine and their derivatives (i.e., amines known to readily condense with ketones to form enamines (Enamines: Synthesis, Structure, and Reactions by A. G. Cook, Marcel Dekker, New York, 1969), combinations of these, and the like. The carboxylic acid may be selected from a wide range of organic acids and may be chiral to help form a chiral halogenated product.

As another option, the salt is provided in situ by using one or more compounds that include both amine functionality and carboxylic acid functionality. Examples of such compounds include one or more amino acids such as L-proline. These form salts in aqueous solution and may be chiral to help form chiral products. Example 3 below describes an alpha halogenation of a monoketal ketone that occurs in the presence of L-proline.

As another approach, a monoketal ketone may be alpha-functionalized with an iodo group by reacting the ketone with a suitable iodine donor. Iodo is very reactive and alpha-functionalization of the monoketal ketone occurs readily in the presence of a suitable iodine functional donor compound. Preferred iodine donor compounds are those that incorporate iodine in a higher oxidation state. An example of one such compound that is commercially available is Iodosylbenzene. lodosylbenzene may also be formed in situ to alpha-halogenate a monoketal ketone from a suitable precursor compound in an alkaline, substantially anhydrous reagent. An example of a suitable precursor is diacetoxyiodobenzene.

Advantageously, the reagent used to convert diacetoxyiodobenzene to iodosylbenzene and then functionalize the ketal ketone with iodo provides those conditions under which a Favorskii rearrangement (discussed further below) occurs. Thus, when the ketone is alpha-functionalized with the iodo group, the desired Favorskii rearrangement then occurs automatically. In practical effect, the conversion of the ketal ketone to the desired ketal acid (such as compound 56 of FIG. 1b) occurs in a single, albeit multi-step, reaction.

Upon completion of the alpha functionalization, the resultant alpha functionalized, cyclic, ketalized ketone may be subjected to conventional work up and isolation procedures. One illustrative work up and isolation procedure adding water the reaction media. This forms separate organic and aqueous phases. Many of the by-products are water-soluble and tend to go into the aqueous phase. The functionalized ketone tends to go into the organic phase such as MTBE (methyl tertiary-butyl ether). The combined organic extracts may then be dried, filtered, and concentrated, as desired.

While these work up and isolation conditions are suitable for obtaining the alpha functionalized product, it is possible that some of the product may be degraded during extractive recovery. For implementation on a larger scale and/or when subjecting the compound to a subsequent Favorskii rearrangement described below, an option would be to eliminate this extractive workup and carry a solution of functionalized ketone directly into the rearrangement.

In another aspect, the present invention relates to subjecting an alpha-functionalized, cyclic, ketalized ketone described herein to a ring contraction reaction. The alpha-functionalized, cyclic, ketalized ketone may be obtained from any suitable source, including via the alpha functionalization reaction scheme(s) described above. In representative reaction schemes the resultant ring-contracted product includes a substituent comprising a carbonyl, —C(O)— moiety. Such substituent may be an ester, acid, salt, amide or other carbonyl derivative.

In the practice of the invention, the Favorskii rearrangement is one illustrative example of a ring contraction reaction scheme that can be applied to convert a cyclic, ketalized, alpha halogenated ketone into a ring contracted, cyclic, ketalized, carbonyl functional product. The Favorskii rearrangement is widely discussed in the technical and patent literature. See, e.g., March et al., March's Advanced Organic Chemistry: Reactions, Mechanisms, and Structure, fifth edition (2001).

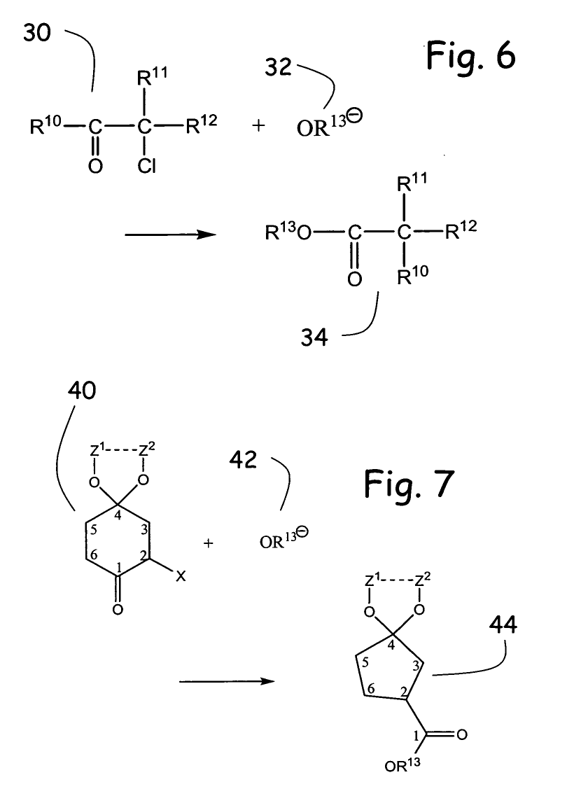

As generally shown in FIG. 6, the Favorskii rearrangement generally involves the reaction of an alpha-halo ketone 30 (e.g., chloro, bromo, or iodo) with alkoxide ion 32 to give a rearranged, carbonyl containing product 34. The R¹⁰, R¹¹, R¹², and R¹³ moieties may be any monovalent moieties, but are desirably free of portions including adjacent keto and leaving groups to avoid generation of rearrangement by-products. Often, the R¹⁰ through R¹³ groups may be linear, branched, and/or cyclic alkyl and/or alkoxy moieties. For purposes of illustration, Cl is shown as the halogen of ketone 30 which is a substituent from one of the carbon atoms at an alpha position relative to the keto group, C(O). In the meantime, the group R10 is at the other alpha position relative to the keto group. For purposes of illustration, the resultant product 34 is shown as an ester. However, depending upon the reaction conditions and steps used, the product 34 may be a carbonyl containing acid, salt or other carbonyl derivative.

While the exact mechanism of the Favorskii reaction is not known with certainty, the result of the rearrangement can be described schematically. Schematically, the Favorskii rearrangement may be viewed as a rearrangement in which the alpha-halogen substituent leaves the ketone 30, resulting in a vacancy at the corresponding alpha carbon. The R¹⁰ moiety migrates from its alpha position to occupy the resultant vacancy left by the leaving halogen. Then, the alkoxide ion 32 occupies the vacancy resulting from the migration of the R¹⁰ moiety. In actuality, it is more likely that the rearrangement may involve a symmetrical, cyclopropanone intermediate as reported at page 1404 of March et al., March's Advanced Organic Chemistry: Reactions, Mechanisms, and Structure, fifth edition (2001).

FIG. 7 schematically shows the general result when the Favorskii rearrangement is applied to the reaction between an illustrative cyclic, ketalized, alpha functionalized ketone 40 and a reactant comprising an alkoxide anion 42 or precursor thereof to form the cyclic carbonyl containing product 44. The source of the alkoxide anion 42 may be an alcohol. For purposes of illustration, product 44 is an ester. In a manner analogous to the Favorskii rearrangement of ketone 30 of FIG. 3, the moiety X is shown for purposes of illustration as the leaving group in the alpha position in ketone 40. For purposes of clarity, only the keto, alpha substituent X, and the ketal group are shown in the reactant. It is understood that the six-membered ring bearing these moieties may also include other substituents such as the R¹ through R⁶ substituents as defined above with respect to the reaction product of FIG. 5. Otherwise, the X, R¹³, Z1, and Z2 moieties are as defined above. The bond between the C6 and C1 carbons of ketone 40 corresponds to the bond between the keto carbon and the R¹⁰ group in FIG. 6.

When the leaving group X leaves the C2 (alpha) carbon at the alpha position of ketone 40, the C6 carbon may be viewed as detaching from the C1 carbon and then attaching to the C2 carbon, occupying the vacancy left by the leaving group. Additionally, the keto group that was part of the cyclic backbone of the reactant becomes a pendant carbonyl substituent in the product. In the meantime, alkoxide anion occupies the resultant vacancy on the newly pendant C1 keto carbon to form the ester moiety.

Note that ketone 40 comprises a six-membered backbone including the C1-C6 carbons. In contrast, the cyclic ester product 44 comprises only a five-membered backbone. Thus, as applied to cyclic alpha-halogenated ketones, the Favorskii rearrangement is an example of a ring contraction reaction.

The Favorskii rearrangement of FIG. 7 preferably is accomplished by heating the ketone reactant 40 in an alkaline, substantially anhydrous solvent. Examples of suitable anhydrous organic solvents include ethanol, methanol, combinations of these and the like. The concentration of the reactant 40 in the solvent can vary over a wide range, although it is desirable that enough solvent be used so that at least substantially all of the reactant is in solution to maximize yield. Yet, although using more solvent than needed to appropriately solvate the reactant could be used if desired, such a practice would waste solvent. Balancing such concerns, using about 1 part by weight of the reactant per about 1 to 10 parts by weight of the solvent would be suitable.

To provide the desired degree of alkalinity, the reaction medium desirably incorporates one or more suitable bases. Examples include NaOH, KOH, sodium carbonate, potassium carbonate, sodium bicarbonate, secondary amines, pyridine, combinations of these, and the like. The concentration of base included in the reaction medium may vary over a wide range. However, if too little is used, then incomplete conversion resulting in mixtures. On the other hand, if too much is used, then the excess base is wasted, and additional acid may be required to later neutralize the excess base. Balancing such concerns, using from about 0.1 to about 5, preferably about 0.5 to about 2 parts by weight of the base per about 1 to 10 parts by weight of the reactant would be suitable.

The rearrangement reaction may be carried out over a wide range of temperatures such as those ranging from about −10° C. to 35° C. to the reflux temperature. Preferably, the reaction medium is heated at reflux to accomplish the rearrangement at a relatively quick rate.

The rearrangement reaction desirably occurs in a protected environment such as those described above. An inert atmosphere of dry N₂ would be one example of a suitable environment.

The product 44 of this Favorskii ring contraction reaction is an ester, which is often hydrolyzed to the acid salt under suitable reaction conditions. This is advantageous inasmuch as salt formation avoids subsequent base-induced condensations of the product 44. Upon completion of the rearrangement reaction, the resultant ring contracted product 44 may be subjected to conventional work up and isolation procedures. In the course of this work up and isolation, the ester may be converted to an acid, salt, or other derivative as desired.

One illustrative work up and isolation procedure involves removing the solvent to leave a residual syrup containing the product. The residual syrup may then be separated between an aqueous phase and an organic phase. The organic extracts contain the neutrals (byproducts and side products). A suitable amount of aqueous acid may then be added to the aqueous phase to lower the pH of the medium to about 4 to 5. In the course of doing this, the acid salt is converted to the acid. The aqueous and organic phases may be extracted with additional organic solvent one or more times. The combined organic extracts containing the acid product may then be dried, filtered, and/or concentrated to recover the product.

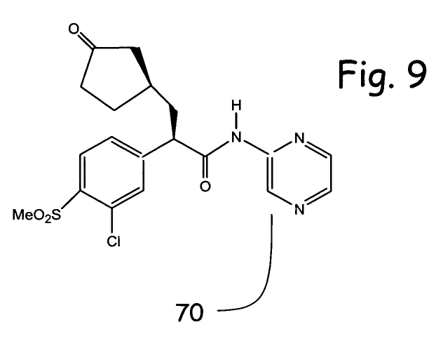

FIG. 1b illustrates the principles of the present invention applied to making the racemic ketal acid salt 50. This compound has been described in German patent documents DE4316576 and DE4312832. The racemic salt 50 may be enantiomerically purified to recover the S-ketal acid salt, which is a useful intermediate in the manufacture of, for instance, a glucokinase activator molecule 70 having the formula shown in FIG. 9. This glucokinase activator molecule 70 is under evaluation in Phase I clinical studies as a potentially new therapy for the treatment of Type 2 diabetes.

In a first reaction step as shown in FIG. 1b, alpha-chloro-functional ketalized ketone 52 is prepared from ketalized ketone 54. The acid ketal 56 is then obtained by subjecting the alpha-chloro-functional ketalized ketone 52 to a Favorskii rearrangement. This acid ketal 56 is then converted to the racemic ketal salt 50 by reaction with S-methylenzylamine (S-MBA).

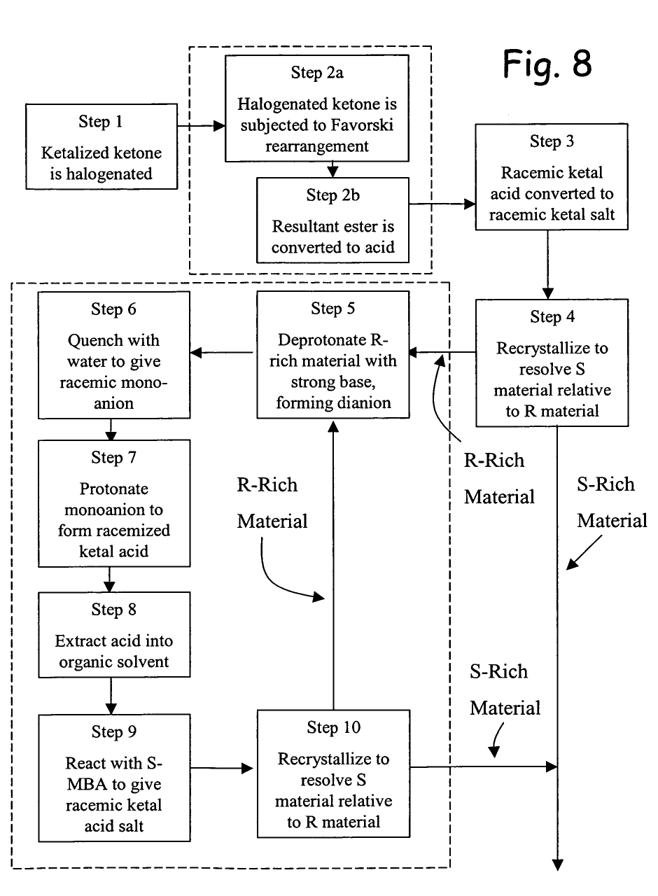

FIG. 8 schematically shows one approach for obtaining the enantiomerically pure chiral (S) salt intermediate 50 of FIG. 1b from the ketalized ketone 54. The chiral S-ketal-acid S-MBA (S-methylenzylamine) salt intermediate has the following structure:

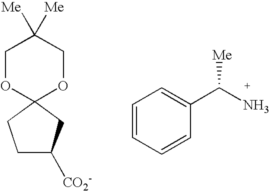

In STEPS 1 and 2, and in accordance with the reaction scheme of FIG. 8, the alpha-chlorinated ketone 52 is prepared from the ketalized ketone 54, and the ketone 52 is subjected to a Favorskii rearrangement and then hydrolyzed to convert the Favorskii ester to the racemic ketal acid 56. The racemic ketal acid is reacted with S-MBA to form the racemic ketal salt in STEP 3:

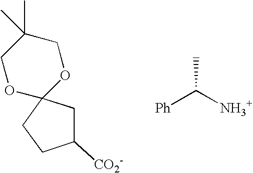

In STEP 4 the racemic ketal salt mixture is recrystallized several times from a suitable solvent mixture in which the R form is more soluble. This allows an increasingly S-rich precipitate to be recovered with each crystallization. One solvent mixture that may be used includes cyclohexane, acetone, and water. The recovered S-rich mixture may then be used, for instance, as an intermediate in substantial, additional synthesis steps that involve modification of the intermediate, followed by reaction with other compounds to build the glucokinase activator molecule 70.

Referring again to FIG. 8, STEP 4 yields not only the S-rich composition but also an R-rich by-product. This R-rich by-product can be racemized with strong base and converted to a racemic ketal acid salt in STEPS 5 through 9 to effectively recycle the undesired R-isomer and any S-isomer that was lost during the original recrystallization. This racemic mixture is subsequently resolved in STEP 10, which is the equivalent operation as carried out in STEP 4. Accordingly, the feed-forward/feedback recycle procedure of STEPS 5 through 10 is intended to accomplish this recovery.

In STEP 5, a strong base is used to deprotonate the chiral carbon of the non-racemic ketal acid salt. The ketal acid salt now exists as an achiral dianion. In STEP 6, water is added to convert the dianion into a racemic, water-soluble carboxylate monoanion.

In STEP 7, the ketal acid monoanion is protonated with an acid to form a racemic ketal acid. The resultant racemic ketal acid is less soluble in aqueous mixtures than the monoanion and is therefore extracted into an organic composition in STEP 8.

In STEP 9, the racemic ketal acid is reacted with S-MBA to form a racemic ketal-acid S-MBA salt. In STEP 10, the racemic mixture is again resolved via multiple recrystallizations to obtain the relatively pure S-ketal salt enantiomer. The S-rich material from STEP 10 is combined with the S-rich material obtained from STEP 4, and the combination of the two S-rich streams is used for GK-2 synthesis. The R-rich by-product from STEP 10 is recycled to STEP 4.

The present invention will now be further described with reference to the following examples.

### EXAMPLE 1

### 3,3-Dimethyl-1,5-dioxaspiro[5.5]undecan-9-one

A continuous extraction apparatus is assembled. A 500 mL extraction solvent pot is charged with 250 mL n-hexane and 5.00 g sodium bicarbonate. An oil bath is heated to 90° C. A 500 mL reaction pot is charged with 82.5 g(0.792 mol, 2.33 equiv) neopentyl glycol, 338 mL H₂O, 0.79 mL (1.45 g, 14.8 mmol, 4.35 mol %) of 98% sulfuric acid, and 38.08 g (0.340 mol) of 1,4-cyclohexanedione. n-Hexane (85 mL) is then added to bring the pot volume to the extractor return sidearm. The extraction pot is immediately immersed in the oil bath and the reaction mixture stir rate is increased to the point where there is efficient mixing in the lower (aqueous) phase but not in the upper (n-hexane) phase in the extractor. The extraction is continued for 99 h.

The suspension is cooled to 25° C. and the precipitate is suction filtered, washed with 50 mL n-hexane, and air dried 2 h at 25° C. to afford 10.71 g of crude bisketal as a colorless solid. The bulk of the n-hexane is distilled from the combined mother liquors and the resulting suspension is cooled (95 g). Methanol (250 mL) is added and 163 mL of a mixture of the methanol-hexane azeotrope (28:72) and methanol are distilled to a head temperature of 60° C. (bath 90° C.). The suspension (168 g) is cooled to 25° C. and water (100 mL) is added dropwise over 10 min. After stirring overnight, the precipitate is suction filtered, and air dried several h at 25° C. to afford 7.22 g of additional crude bisketal as a colorless solid.

The mother liquors are concentrated by distillation (dry ice-acetone cold finger condenser) at 30-35° C. and 40-45 mm Hg (146 mL distillate collected). The resulting suspension is cooled to 0-5° C. and stirred for 90 min. The precipitate is suction filtered (mother liquors are used to complete the transfer) and air dried 24 h at 25° C. to afford 50.17 g (74.5%) of the monoketal 54 as a colorless solid.

The combined crude bisketal crops (17.62 g) are resuspended in 200 mL water and stirred for 1 h. The insoluble material is suction filtered and air dried 6 h at 25° C. to afford 13.06 g of bisketal as a colorless solid.

### EXAMPLE 2

### 8,8-Dimethyl-6,10-dioxaspiro[4,5]decane-2-carboxylic acid

In a foil-covered flask, a solution of 1.000 g (5.04 mmol) of the monoketal of Example 1 and 0.674 g (5.04 mmol) of N-chlorosuccinimide in 1.0 mL dry DMF was stirred at 25° C. for 69 h. With the lab lights off, water (10 mL) was added and the mixture extracted with 5 mL MTBE five times. The combined MTBE extracts were dried (MgSO₄), filtered, and concentrated in vacuo (rotary evaporator at 30° C. and 100 mm Hg then vacuum pump at 25° C. and 1 mm Hg for 30 min) to afford 1.135 g of crude chloroketone product as a pale yellow solid.

An ethanolic KOH solution was prepared by dissolving 1.14 g (17.3 mmol) of 85% KOH pellets in 5.0 mL anhydrous ethanol at 70° C. A solution of 1.135 g (˜4.88 mmol) of crude chloroketone in 7.0 mL of anhydrous ethanol was then added dropwise to the ethanolic KOH solution at 70° C. over 12 min. The resulting suspension was refluxed for 1 h (bath 80° C.)(dry N₂).

The suspension was cooled and ethanol removed on a rotary evaporator at 30° C. and 40 mm Hg. The residual syrup was separated between 5 mL H₂O and 5 mL MTBE. The aqueous layer was extracted with 5 mL MTBE twice more. These extracts contain the neutrals.

MTBE (5 mL) was added followed by 1.0 M aqueous citric acid (7.0 mL) to bring the pH to 4-5. The layers were separated. The aqueous layer was extracted with 5 mL MTBE five times. These extracts contain the resultant carboxylic acid. The combined organic extracts containing carboxylic acid were dried (MgSO₄), filtered, and concentrated in vacuo (rotary evaporator at 30° C. and 100 mm Hg then vacuum pump at 25° C. and 1 mm Hg for 15 h) to afford 679 mg (62.8%) of compound 56 as tan solid.

### EXAMPLE 3

### 8,8-Dimethyl-6,10-dioxaspiro[4,5]decane-2-carboxylic acid

A mixture of 10.00 g (50.44 mmol) of the monoketal of Example 1,7.072 g (53.0 mmol, 1.05 equiv) of N-chlorosuccinimide, 581 mg 95.04 mmol, 10 mol%) of L-proline, and 50 mL isopropanol was stirred at −5° C. for 21.5 h to produce a suspension of crude chloroketone.

A solution of 15.02 g (227.6 mmol) of 85% potassium hydroxide in 60 mL anhydrous ethanol was prepared at 70° C. The suspension of crude chloroketone was then added via Teflon cannula over 20 min at 70° C. The resulting suspension was refluxed (bath 80° C.) for 1 h.

The suspension was cooled and solvents removed on a rotary evaporator at 30° C. and 50-40 mm Hg. The residue was taken up in 50 mL H₂O, washed with 50 mL toluene twice, and washed with 25 mL MTBE three times. The suspension was then added to 300 mg of 18 wt % palladium hydroxide on carbon and the suspension hydrogenated at 25° C. and 36-32 psi H₂ for 17 h. The catalyst was removed by filtration through celite. Toluene was added to the mother liquor. Citric acid (70 ml of 2 M) was added to reduce the pH to 4. The layers were separated and the aqueous layer extracted with 25 mL toluene four more times. The combined extracts were dried (MgSO4), filtered, and concentrated in vacuo (rotary evaporator at 30 C and 25 mm Hg then vacuum pump at 25 C and 1 mm Hg for 4 h) to afford 6.59 g (61.0%) of compound 56 as a tan solid.

### EXAMPLE 4

### 8,8-Dimethyl-6,10-dioxaspiro[4,5]decane-2-carboxylic acid

A solution of 1.33 g (20.2 mmol) of 85% potassium hydroxide pellets in 10 mL methanol was prepared and cooled in a water bath. The ketone (1.000, 5.04 mmol) of the monoketal of Example 1, was added followed by 1 mL methanol. The yellow solution was stirred at 25° C. for 60 sec. Diacetoxyiodobenzene (1.625 g, 5.04 mmol) was added followed by 1 mL methanol. The solution was stirred at 25° C. for 1 h. Under these reactions conditions, iodosylbenzene is formed, which serves as a donor compound to alpha-functionalize the ketal ketone with iodo functionality. Also, the reaction medium used to functionalize the ketal ketone with iodo functionality generally provides the conditions to carry out a Favorskii rearrangement reaction. Consequently, once the alpha-iodo-functionalized material is formed, the material automatically proceeds to rearrange according to the Favorskii scheme.

Methanol was removed on a rotary evaporator at 30° C. and 70 mm Hg. The residue was separated between 15 mL water and 15 mL toluene. The aqueous layer was washed with 15 mL toluene four more times. Toluene (15 mL) was added to the aqueous layer followed by 2 M citric acid (10 mL) to reduce the pH to 4. The layers were separated and the aqueous layer extracted with 15 mL toluene four more times. The combined post-acid toluene extracts were dried (MgSO₄), filtered, and concentrated in vacuo (rotary evaporator at 30° C. and 25 mm Hg then vacuum pump at 25° C. and 15 mm Hg for 3 h) to afford 0.757 g (70.0%) of compound 56 as a pale yellow solid.

All patents, patent applications, technical articles and books cited herein are incorporated herein by reference in their respective entireties for all purposes.

What is claimed is:

1. A compound, comprising:
a) a cyclic moiety comprising a backbone of at least 4 atoms and having first and second alpha positions adjacent a keto group;
b) at least one hydrogen substituent positioned at the first alpha position;
c) a leaving group substituent positioned at the second alpha position; and
d) a ketal substituent positioned at a third position that is at a beta position or further from the keto group.

2. The compound of claim 1, wherein the cyclic moiety comprises a backbone of 5 atoms.

3. The compound of claim 1, wherein the cyclic moiety comprises a backbone of 6 atoms.

4. The compound of claim 1, wherein the keto group is part of the backbone and associated with a C1 position of the cyclic moiety and wherein the ketal substituent is part of the backbone and associated with a C4 position of the cyclic moiety.

5. The compound of claim 1, wherein the leaving group is selected from Cl, I, Br, and OH.

6. The compound of claim 1, wherein the leaving group is Cl.

7. The compound of claim 1, wherein the leaving group is I.

8. The compound of claim 1, wherein the compound further comprises at least one additional substituent selected from hydrogen; linear, branched, or cyclic alkyl; alkoxy; aryl; and combinations of these; and wherein the compound is free of additional substituents selected from a ketone, nitro, and aldehyde.

9. The compound of claim 1, wherein the compound has the structure 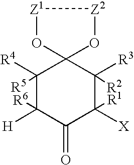
wherein X is a leaving group; each of Z¹ and Z² independently represent a monovalent group, or as represented by the dashed line, are co-members of a ring structure providing a divalent moiety -Z¹-Z²-; and each of R¹ through R⁶ substituents independently represents a monovalent group or any two of the R¹ through R⁶ substituents are co-members of a ring structure.

10. The compound of claim 9, wherein Z¹ and Z² are co-members of a ring structure.

11. The compound of claim 10, wherein said ring structure has the formula
—CH₂—C(CH₃)₂—CH₂—

12. The compound of claim 10, wherein each of R¹ through R⁶ is hydrogen.

13. The compound of claim 10, wherein X is selected from Cl, Br, I, and OH.

14. The compound of claim 10, wherein X is selected from I.

15. The compound of claim 1, wherein the compound has the formula

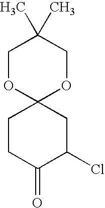

16. The compound of claim 10, wherein each of the R¹-R⁶ substituents independently is (1) a monovalent substituent selected from H, R, OR, or aryl, wherein R is a linear, branched or cyclic alkyl, alkoxy, or aryl moiety, or (2) a co-member of a ring structure with at least one of the other R¹-R⁶ substituents.

17. The compound of claim 10, wherein each of the R¹-R⁶ substituents independently is (1) a monovalent substituent other than a keto group, an aldehyde group, a nitro group, or another group that is reactive with ketone in an alkaline environment, or (2) a co-member of a ring structure with at least one of the other R¹-R⁶ substituents.

18. A method of alpha-halogenating a ketone compound, comprising the steps of:
a. providing a ketone compound, comprising:
i. a cyclic moiety comprising a backbone of at least 4 atoms and having first and second alpha positions adjacent a keto group;
ii. at least one hydrogen substituents positioned at the first alpha position;
iii. a leaving group substituent positioned at the second alpha position; and
iv. a ketal or acetal substituent positioned at a third position that is at a beta position or further from the keto group;
b. providing a donor compound; and
c. reacting ingredients comprising the compounds of steps (a) and (b) in a substantially anhydrous solvent that is sufficiently polar so that alpha-functionalization of the keto compound occurs.

19. The method of claim 18, wherein the substantially anhydrous solvent comprises DMF.

20. The method of claim 18, wherein step (c) occurs at a temperature in the range of −10° C. to 35° C.

21. The method of claim 18, wherein step (c) occurs in the absence of added acid and base.

22. The method of claim 18, wherein the donor compound comprises a moiety of the formula —C(O)—N(X)—, wherein X is selected from Cl, Br, I, and OH.

23. The method of claim 18, wherein the donor compound is selected from N-chlorosuccinimide, dichlorodimethylhydantoin, trichloroisocyanurate, and combinations of these.

24. The method of claim 18, wherein the donor compound is N-chlorosuccinimide.

25. The method of claim 18, wherein the donor compound is water soluble.

26. The method of claim 18, wherein the keto compound has the formula 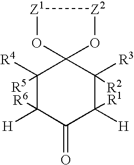
wherein each of Z¹ and Z² independently represent a monovalent group, or as represented by the dashed line, are co-members of a ring structure providing a divalent moiety -Z¹-Z²-; and each of R¹ through R⁶ substituents independently represents a monovalent group or any two of the R¹ through R⁶ substituents are co-members of a ring structure.

27. The compound of claim 26, wherein Z¹ and Z² are co-members of a ring structure.

28. The compound of claim 27, wherein said ring structure has the formula
—CH₂—C(CH₃)₂—CH₂—

29. The compound of claim 26, wherein each of R¹ through R⁶ is hydrogen.

30. The compound of claim 26, wherein the keto compound has the formula

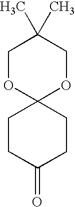

31. The compound of claim 26, wherein each of the R¹-R⁶ substituents independently is (1) a monovalent substituent selected from H, R, OR, or aryl, wherein R is a linear, branched or cyclic alkyl, alkoxy, or aryl moiety, or (2) a co-member of a ring structure with at least one of the other R¹-R⁶ substituents.

32. The compound of claim 26, wherein each of the R¹—R⁶ substituents independently is (1) a monovalent substituent other than a keto group, an aldehyde group, a nitro group, or another group that is reactive with ketone in an alkaline environment, or (2) a co-member of a ring structure with at least one of the other R¹-R⁶ substituents.

33. A method of halogenating a ketalized ketone, comprising the step of halogenating the ketone in an anhydrous, organic reagent in the presence of a salt catalyst, wherein the reagent comprises an alcohol.

34. The method of claim 33, wherein the salt is obtained from one or more ingredients comprising amine functionality and carboxylic acid functionality.

35. The method of claim 33, further comprising the step of forming the salt catalyst in situ.

36. The method of claim 33, wherein the salt is formed in situ from an ingredient comprising amine and carboxylic acid functionalities.

37. The method of claim 33, further comprising the step of subjecting the halogenated ketone to a Favorskii rearrangement.

38. A method of making a ketal acid comprising reacting a ketalized ketone with an iodine donor compound in an alkaline reaction medium.

39. The method of claim 38, wherein the alkaline reaction medium is substantially anhydrous.

40. The method of claim 38, wherein the iodine donor compound is iodosylbenzene or a precursor thereof.

41. The method of claim 38, wherein the ketalized ketone has the formula 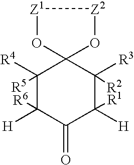
wherein each of Z¹ and Z² independently represent a monovalent group, or as represented by the dashed line, are co-members of a ring structure providing a divalent moiety -Z¹-Z²-; and each of R¹ through R⁶ substituents independently represents a monovalent group or any two of the R¹ through R⁶ substituents are co-members of a ring structure.

42. A method of making a compound comprising the steps of:
a) halogenating a ketalized, cyclic ketone at an alpha position relative to a keto group; and
b) subjecting the halogenated, ketalized cyclic ketone to a ring contraction reaction.

43. The method of claim 42, wherein the ring step (b) comprises heating the halogenated, ketalized cyclic ketone in an alkaline, substantially anhydrous solvent in the presence of an alkoxide ion.

44. The method of claim 43 wherein a source of the alkoxide ion is an alcohol.

45. The method of claim 42, wherein the cyclic, ketalized ketone has the formula 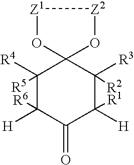
wherein each of Z¹ and Z² independently represent a monovalent group, or as represented by the dashed line, are co-members of a ring structure providing a divalent moiety -Z¹-Z²-; and each of R¹ through R⁶ substituents independently represents a monovalent group or any two of the R¹ through R⁶ substituents are co-members of a ring structure.

46. The compound of claim 45, wherein Z¹ and Z² are co-members of a ring structure.

47. The compound of claim 46, wherein said ring structure has the formula
—CH₂—C(CH₃)₂—CH₂—

48. The compound of claim 45, wherein each of R¹ through R⁶ is hydrogen.

49. The compound of claim 45, wherein the keto compound has the formula

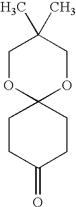

50. The compound of claim 45, wherein each of the R¹-R⁶ substituents independently is (1) a monovalent substituent selected from H, R, OR, or aryl, wherein R is a linear, branched or cyclic alkyl, alkoxy, or aryl moiety, or (2) a co-member of a ring structure with at least one of the other R¹-R⁶ substituents.

51. The compound of claim 45, wherein each of the R¹-R⁶ substituents independently is (1) a monovalent substituent other than a keto group, an aldehyde group, a nitro group, or another group that is reactive with ketone in an alkaline environment, or (2) a co-member of a ring structure with at least one of the other R¹-R⁶ substituents.
